(19) United States
(12) Patent Application Publication
(10) Pub. No.: US 2025/0311976 A1
(43) Pub. Date: Oct. 9, 2025

(54) SYSTEMS AND METHODS FOR IDENTIFYING POSITION OF ELECTRODES IN AN EEG ELECTRODE ARRAY ON A HEAD OF A SUBJECT

(71) Applicant: HADASIT MEDICAL RESEARCH SERVICES AND DEVELOPMENT LTD., Jerusalem (IL)

(72) Inventors: Mordehay MEDVEDOVSKY, Jerusalem (IL); Evgeny TSIZIN, Jerusalem (IL); Dana EKSTEIN, Jerusalem (IL)

(21) Appl. No.: 18/865,631

(22) PCT Filed: May 15, 2023
(86) PCT No.: PCT/IL2023/050502

§ 371 Date: Nov. 13, 2024

Related U.S. Application Data
(60) Provisional application No. 63/342,201, filed on May 16, 2022.

(51) Int. Cl. A61B 5/00; A61B 5/256; A61B 5/291; G06T 7/00; G06T 7/73

(52) U.S. Cl. CPC A61B 5/6841; A61B 5/256; A61B 5/291; A61B 5/6803; G06T 7/0002; G06T 7/74; G06T 7/75; A61B 2090/363; A61B 2560/0468; A61B 2562/046; G06T 2207/10004; G06T 2207/20081; G06T 2207/30204

(57) ABSTRACT

Disclosed herein are methods and systems for identifying a position of a plurality of electrodes in an EEG electrode array embedded to an EEG electrode array carrier when the EEG electrode array carrier is worn on a head of a subject. A first system is an optical system containing a stereo camera pair which captures at least one image of the head of the subject wearing the EEG electrode array carrier. A second system is an electrical system which constructs a 3D electrical model of the EEG electrode array and an electrical model of a couplant spreading of the couplant which couples the electrodes to the head of the subject. The two systems may be integrated to one electro-optical system for identifying a position of electrodes in an EEG electrode array on a head of a subject.

### TECHNICAL FIELD

The present disclosure, in some embodiments thereof, relates to EEG systems. More particularly, but not exclusively, to systems and methods for identifying the position of electrodes in an EEG electrode array on a head of a subject.

### BACKGROUND

Electroencephalography (EEG) is a method for recording the electrical activity of the brain. The EEG procedure is traditionally managed and carried out by special technicians and is used for detecting and diagnosing various brain problems, for example, epilepsy. During the EEG procedure, a plurality of electrodes, which are connected by wires to an EEG recording system are coupled to the subject's head, usually through a couplant such as gel or paste. To analyze the EEG records it is important to know the exact locations of the electrode-skin contacts on the subject's head. The technicians performing the EEG tests are qualified to place the electrodes in specific positions on the head of the subject and the tests are conducted in a clinical environment.

In some cases, it is required to perform the EEG test during an extended period of time, for example during a few weeks. Since the tests are carried out in a clinical environment by a technician, such cases become very expensive and involve a great extent of discomfort for the patients. EEG systems are rarely used at home due to the challenge of positioning the EEG electrodes in the correct positions every time the electrode array is worn by the subject. For a non-qualified person, positioning the electrodes in the correct positions on the head is difficult, cumbersome, and challenging.

One currently used method for determining the positions of the electrodes on the head of the subject utilizes a three-dimensional (3D) scanning. In this case, the first test is carried out by a technician, who identifies the positions for the electrodes and couples the electrodes correctly to the head of the subject. Then the head of the subject is scanned using a scanning technique, for example, an optical scan of the head by taking pictures of the head of the subject wearing the electrodes from several different angles and constructing a 3D model of the head of the subject. In the following EEG tests, the positions of the EEG electrodes worn by the subject need to be re-determined. A first option is to estimate the position of the electrodes by performing a 3D scanning, typically by taking multiple pictures of the head of the subject wearing the EEG electrode array from different angles and reconstructing a 3D model. However, during the process of taking the pictures from different angles, the subject may move and interfere with the reconstruction process of the 3D model, in which case the process needs to be repeated until the subject is still enough to enable proper scanning process. This method is thus time consuming and cumbersome particularly when the EEG test must be repeated daily.

Another possibility is to use a system consisting of many multiple synchronized cameras (about 15 cameras). Such a system solves the problem of the subject movement during the scanning however, it is cumbersome and overly expensive for home use.

Thus, there is a need for a system and method for allowing a simple and reliable positioning of an EEG electrode array that may be applied by a non-qualified user and outside of a clinic or a hospital.

### SUMMARY

According to some embodiments, provided herein are systems and methods for identifying the positions of a plurality of electrodes in an EEG electrode array embedded to an EEG electrode array carrier worn on a head of a subject, by using an optical system containing a stereo camera pair which captures at least one image of the head of the subject wearing the EEG electrode array carrier, thereby enabling the system which is directed to home use to be cost effective and easy to use.

According to some embodiments, a first system is an optical system containing a stereo camera pair which captures at least one image of the head of the subject wearing the EEG electrode array carrier. According to some embodiments, a second system is an electrical system which constructs a 3D electrical model of the EEG electrode array and an electrical model of a couplant spreading of the couplant which couples the electrodes to the head of the subject. The two systems may be integrated to one electro-optical system for identifying a position of electrodes in an EEG electrode array on a head of a subject.

According to some embodiments, provided herein are systems and methods for identifying the positions of a plurality of electrodes in an EEG electrode array embedded to an EEG electrode array carrier worn on a head of a subject, by using an electrical system which constructs a 3D electrical model of the EEG electrode array and an electrical model of a couplant spreading of the couplant which couples the electrodes to the head of the subject. These electrical methods and systems when used together with the optical system provide highly accurate results on the one hand yet keeps the system to be cost effective and simple to use on the other hand.

According to some embodiments, provided herein are systems and methods for identifying the position of the electrodes in the EEG electrode array relative to the head when worn on the head of the subject, and for compensating for the discrepancies detected when the electrodes are positioned in a wrong position.

Thus according to one aspect there is provided herein a method for identifying a position of a plurality of electrodes in an EEG electrode array embedded to an EEG electrode array carrier when the EEG electrode array carrier is worn on a head of a subject, the method comprises capturing at least one image of three fiducials of the subject's head and at least two electrodes or visible marks around electrodes placed at known positions of the EEG electrode array carrier worn by the subject, using a stereo camera pair;
defining a coordinate system of the three fiducials and relating the at least two electrodes or visible marks around electrodes to the defined coordinate system;
determining the position of each of the plurality of electrodes on the head of the subject wearing the EEG electrode array carrier based at least on:
   the defined coordinate system and triangulation of the three fiducials and at least two electrodes or visible marks around electrodes of the EEG electrode array carrier, and
   a previously obtained 3D geometrical model of the subject's head and of the EEG electrode array, and a known mechanical model of the EEG electrodes' array carrier thereby, reconstructing a 3D geometrical model of the EEG electrode array embedded to the EEG electrode array carrier worn on the subject's head.

According to some embodiments, the method, further comprises: injecting current into one or more electrodes of the EEG electrode array, coupled to the head of the subject through a couplant;
measuring the voltage response on other electrodes of the EEG electrode array;
constructing a 3D electrical model of the EEG electrode array embedded to the EEG electrode array carrier worn on the subject's head;
constructing an electrical model of a couplant spreading based at least on:
   the voltage response as a function of the shape of the couplant spreading of all the electrodes of the EEG electrode array over the subject's head surface, and
   the 3D electrical model of the EEG electrode array embedded to the EEG electrode array carrier worn on the subject's head; and
identifying the weight center of the couplant coupling the electrodes, according to the electrical model of the couplant spreading, thereby identifying the position of the electrodes.

According to some embodiments, the method further comprises coupling dry electrodes to the head of the subject and identifying the position of said dry electrodes according to a capacitive coupling function derived from the 3D electrical model of the EEG electrode array.

According to some embodiments, capturing the at least one image of three fiducials and at least two electrodes or visible marks around electrodes placed at known positions of the EEG electrode array carrier is done using a multi-view camera system, which captures at least one multi-view set of images at least at a single time moment.

According to some embodiments, capturing at least one image of three fiducials of the subject's head and at least two electrodes or visible marks around electrodes placed at known positions of the EEG electrode array carrier, is done using one calibrated camera.

According to some embodiments, when using the one calibrated camera, determining the position of each of the plurality of electrodes on the head of the subject wearing the EEG electrode array carrier is based on position estimation of the three fiducials and at least two electrodes or visible marks around electrodes of the EEG electrode array carrier instead of triangulation thereof.

According to some embodiments, the previously obtained 3D geometrical model of the subject's head and of the EEG electrode array is obtained by previously wearing by the subject the EEG electrode array carrier and measuring the geometrical model by a technician.

According to some embodiments, the mechanical model of the EEG electrodes' array carrier, is un-stretchable, and preserves the geodetic distances between the electrodes.

According to some embodiments, the mechanical model of the EEG electrodes' array carrier, is stretchable, with a known value of elasticity.

According to some embodiments, the method further comprises:
successively capturing one or more additional images of the head of the subject wearing the EEG electrode array carrier so that at least three elements out of the three fiducials and the at least two electrodes or visible marks around electrodes in known positions of the EEG electrode array carrier, from the at least one image or from one of the multi-view set of images which are used as a reference image are visible. Then, relating electrodes which are visible in the one or more additional images to the coordinate system defined in the reference image, and triangulating other electrodes visible in the one or more additional images, thereby identifying the position of the electrodes visible in the one or more additional images.

According to some embodiments, the camera is a video camera or a still camera.

According to some embodiments, the EEG electrode array carrier is a cap or a net.

According to some embodiments, the electrical models of the EEG electrode array and of the couplant spreading are used as a reference for the reconstructed 3D geometrical model of the EEG electrode array, thereby constructing a 3D electro-geometrical model of the EEG electrode array embedded to the EEG electrode array carrier worn on the subject's head.

According to some embodiments, the method further comprising:
detecting a potential wrinkle in or a displacement of the EEG electrode array carrier worn by the subject by capturing one or more additional images of the head of the subject wearing the EEG electrode array carrier and/or by executing a machine learning algorithm by a processor, on the constructed 3D electro-geometrical model of the EEG electrode array; and
if a wrinkle or a displacement of the EEG electrode array carrier is detected, providing indication to the subject to realign the EEG electrode array carrier on their head or compensating for the wrinkle or displacement of the EEG electrode array carrier upon constructing the 3D electro-geometrical model of the EEG electrode array.

According to some embodiments, the machine learning algorithm is a deep neural network trained on labeled database records of wrinkles.

According to some embodiments, the database records of wrinkles are optical and/or electrical records.

According to some embodiments, the successive capturing of the one or more additional images of the head of the subject wearing the EEG electrode array carrier is done when the subject's head is rotated relative to the position of the head when capturing the al least one image or when the stereo camera pair or multi-view camera system or one calibrated camera is rotated around the subject's head.

According to some embodiments, the previously obtained 3D geometrical model of the subject's head is constructed according to at least one of the following: a set of photographs, video, Magnetic Resonance Imaging (MRI) scanning or Computed Tomography (CT) scanning of the subject's head and scanning of the subject's head with the EEG electrode array carrier.

According to some embodiments, the spreading of the couplant is measured through electrodes which are not initially coupled to the subject's head, but due to the spreading of the couplant said electrodes got connected to couplant.

According to another aspect, there is disclosed herein a method for identifying a position of a plurality of electrodes in an EEG electrode array embedded to an EEG electrode array carrier when the EEG electrode array carrier is worn on a head of a subject, the method comprises:
injecting current into one or more electrodes of the EEG electrode array, coupled to the head of the subject through a couplant;
measuring the voltage response on other electrodes of the EEG electrode array;
constructing a 3D electrical model of the EEG electrode array embedded to the EEG electrode array carrier worn on the subject's head;
constructing an electrical model of a couplant spreading based at least on:
   the voltage response as a function of the shape of the couplant spreading of all the electrodes of the EEG electrode array over the subject's head surface, and
   the 3D electrical model of the EEG electrode array embedded to the EEG electrode array carrier worn on the subject's head; and
identifying the weight center of the couplant coupling the electrodes, according to the electrical model of the couplant spreading, thereby identifying the position of the electrodes.

According to another aspect, there is disclosed herein a system for identifying a position of a plurality of electrodes in an EEG electrode array embedded to an EEG electrode array carrier when the EEG electrode array carrier is worn on a head of a subject, the system comprises:
a stereo camera pair, capturing at least one image of three fiducials of the subject's head and at least two electrodes or visible marks around electrodes placed at known positions of the EEG electrode array carrier worn by the subject;
a display for presenting EEG signals received from the EEG electrode array carrier and for locating the stereo camera pair; and
a processor, executing a code for:
   defining a coordinate system of the three fiducials and relating the at least two electrodes or visible marks around electrodes placed at known positions of the EEG electrode array carrier to the defined coordinate system; and
   determining the position of each of the plurality of electrodes on the head of the subject wearing the EEG electrode array carrier based at least on:
      the defined coordinate system and triangulation of the three fiducials and at least two electrodes or visible marks around electrodes of the EEG electrode array carrier, and
      a previously obtained 3D geometrical model of the subject's head and of the EEG electrode array, and a known mechanical model of the EEG electrodes' array carrier thereby, reconstructing a 3D geometrical model of the EEG electrode array embedded to the EEG electrode array carrier worn on the subject's head.

According to some embodiments, the system further comprises:
an electrical power source for injecting current into one or more electrodes of the EEG electrode array, coupled to the head of the subject through a couplant;
a voltmeter for measuring the voltage response on other electrodes of the EEG electrode array;
a processor executing a code for:
   constructing a 3D electrical model of the EEG electrode array embedded to the EEG electrode array carrier worn on the subject's head;
   constructing an electrical model of a couplant spreading based at least on:
      the voltage response as a function of the shape of the couplant spreading of all the electrodes of the EEG electrode array over the subject's head surface, and
      the 3D electrical model of the EEG electrode array embedded to the EEG electrode array carrier worn on the subject's head; and
   identifying the weight center of the couplant coupling the electrodes, according to the electrical model of the couplant spreading, thereby identifying the position of the electrodes.

According to some embodiments, the system further comprises:
capsules with a constant amount of couplant which are embedded to the EEG electrode array carrier wherein each capsule is located near one electrode of the EEG electrode array.

According to some embodiments, electrodes of the EEG electrode array are located inside the capsules, as electrode-capsule, and when a capsule is opened, the couplant inside the capsule moves to the space between the electrode inside the capsule and the head of the subject.

According to some embodiments, the EEG electrode array carrier used with the capsules or electrode-capsules is a transparent cap or a cap with one or more transparent windows.

According to some embodiments, the transparent cap or one or more transparent windows comprise a marked border to indicate recommended borders of couplant smearing.

According to some embodiments, the stereo camera pair captures at least one image of the subject's head wearing the transparent cap or the cap with one or more transparent windows after the couplant is smeared on the subject's head, and the processor executes a code for determining the couplant edges based on the at least one image captured.

According to some embodiments, the couplant edges is determined based on electrically measured intercontact distances, and a degree of compatibility between the couplant edges determined based on the at least one image and the couplant edges determined based on the electrically measured intercontact distances, is estimated.

According to some embodiments, parameters of source localization are determined based on the degree of compatibility between the couplant edges determined based on the at least one image and the couplant edges determined based on the electrically measured intercontact distances.

According to some embodiments, the system further comprises electrode-stickers which are embedded to the EEG electrode array carrier wherein the electrode-capsules are coupled to locations with hair on the subject's head and the electrode-stickers are coupled to locations without hair.

According to another aspect, there is disclosed herein a system for identifying a position of a plurality of electrodes in an EEG electrode array embedded to an EEG electrode array carrier when the EEG electrode array carrier is worn on a head of a subject, the system comprises:
an electrical power source for injecting current into one or more electrodes of the EEG electrode array, coupled to the head of the subject through a couplant;
a voltmeter for measuring the voltage response on other electrodes of the EEG electrode array;
a processor executing a code for:
constructing a 3D electrical model of the EEG electrode array embedded to the EEG electrode array carrier worn on the subject's head
constructing an electrical model of a couplant spreading based at least on:
the voltage response as a function of the shape of the couplant spreading of all the electrodes over the head surface, and
the 3D electrical model of the EEG electrode array embedded to the EEG electrode array carrier worn on the subject's head; and
identifying the weight center of the couplant coupling the electrodes, according to the electrical model of the couplant spreading, thereby identifying the position of the electrodes.

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the figures:

FIG. 1 schematically shows a block diagram of a system 100, for identifying position of a plurality of electrodes in an EEG electrode array embedded to an electrode array carrier when the EEG electrode array carrier is worn on a head of a subject, according to some embodiments;

FIG. 2 schematically shows an example of a system 200 for identifying a position of a plurality of electrodes in an EEG electrode array embedded to an electrode array carrier when the EEG electrode array carrier is worn on a head of a subject, according to some embodiments;

FIGS. 3a-3f schematically shows an example of one image used as a reference image (FIG. 3a) with three fiducials and two electrodes visible, and additional images successively captured (FIGS. 3b-3e) with at least three elements visible out of the three fiducials and the two electrodes, according to some embodiments;

FIG. 4 schematically shows a block diagram of an electro-optical system 400 where an electrical system is connected to the optical system for identifying the position of the electrodes in an EEG electrode array embedded to an electrode array carrier when the EEG electrode array carrier is worn on a head of a subject, according to some embodiments;

FIG. 5 schematically shows an electrical system 500 for identifying the position of electrodes in an EEG electrode array embedded into an EEG electrode array carrier, worn by a subject, according to some embodiments;

FIGS. 6a-6f schematically show an example for an electrical system according to some embodiments and results of exemplary cases experiments of proper and improper spreading of the couplant coupling the electrodes in the EEG electrode array to a skin of a subject, according to some embodiments;

FIG. 7 schematically shows a flow chart of a method for identifying a position of a plurality of electrodes in an EEG electrode array embedded to an EEG electrode array carrier when the EEG electrode array carrier is worn on a head of a subject, according to some embodiments;

FIG. 8 schematically shows a flow chart of a method for identifying a position of a plurality of electrodes in an EEG electrode array embedded to an electrode array carrier when the EEG electrode array carrier is worn on a head of a subject, by successively capturing additional images of the head of the subject wearing the EEG electrode array carrier, according to some embodiments;

FIG. 9 schematically shows a flow chart of an electrical method for identifying a position of a plurality of electrodes in an EEG electrode array embedded to an electrode array carrier when the EEG electrode array carrier is worn on a head of a subject, according to some embodiments;

FIG. 10 schematically shows a flow chart of an electro-optical method for identifying a position of a plurality of electrodes in an EEG electrode array embedded to an electrode array carrier when the EEG electrode array carrier is worn on a head of a subject, when a wrinkle is detected in the electrode array carrier or a displacement of the EEG electrode array carrier is detected, according to some embodiments;

FIGS. 11a-11c show three examples of prototypes for a structure of an optical experimental system, according to some embodiments;

FIG. 12 shows an example of a final prototype of an optical system where the final prototype of the structure of FIG. 11c is connected to a processor and a screen, according to some embodiments;

FIG. 13a shows an example of a phantom head with openings, according to some embodiments;

FIG. 13b shows EEG electrodes, including reference, ground and other electrodes connected to a plate containing a mixed soft conductive material (SCM), according to some embodiments;

FIG. 13c shows the phantom head smeared with the SCM of FIG. 13b, according to some embodiments;

FIG. 14 shows an example of capsules of SCM placed on the back part of the phantom head and EEG electrodes (ground, reference, and other electrodes) inserted into these capsules and connected to an EEG device (“Natus®”, not shown), according to some embodiments;

FIG. 15 shows an example of a user interface screen of a Function Generator application, according to some embodiments;

FIG. 16a shows results of voltage measurements between electrodes T4 and T6 before paste smearing, according to some embodiments;

FIG. 16b shows results of voltage measurements between electrodes T4 and T6 after paste smearing according to some embodiments;

FIG. 17a shows results of a furrier transform of channel T4-T6 before paste smearing, according to some embodiments;

FIG. 17b shows results of a furrier transform of channel T4-T6 after paste smearing, according to some embodiments;

FIG. 18a shows an example of a periauricular sticker-electrodes attached to a head of a subject, according to some embodiments;

FIG. 18b shows an example of periauricular sticker-electrodes and a side view of frontal sticker-electrodes attached to a head of a subject, according to some embodiments;

FIG. 18c shows an example of a front view of frontal sticker-electrodes attached to a head of a subject, according to some embodiments;

FIG. 18d shows an example of a combination of a frontal sticker-electrode set and a capsule-electrode cap attached to the head of a subject, according to some embodiments;

FIGS. 19a-19b schematically show EEG traces during drowsiness and wakefulness respectively, using capsule electrodes with broad contacts, according to some embodiments;

FIG. 20a shows an example of conductive paste capsules 2001 smeared as thin disk-like shapes, according to some embodiments;

FIG. 20b shows an example of a transparent window in an EEG cap and capsule electrode before paste smearing, according to some embodiments; and

FIG. 20c shows an example of a transparent window in an EEG cap and capsule electrode after paste smearing, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, provided herein are advantageous systems and methods for identifying a position of a plurality of electrodes in an EEG electrode array embedded to an EEG electrode array carrier when the EEG electrode array carrier is worn on a head of a subject. These advantageous systems and methods may be used in a domestic environment at a nonclinical environment and may ease the use the EEG system for users that are non-qualified (i.e., users which are not EEG technician) using the system at home.

According to some embodiments, as used herein, the terms “optical model”, and “geometrical model” may interchangeably be used. The terms are directed to a 3D model of an EEG electrode array embedded to an EEG electrode array carrier worn on a head of a subject which is constructed based on an optical scanning of the head of the subject wearing the EEG electrode array carrier, by one or more cameras, which provides the geometrical position of the electrodes in the EEG electrode array.

According to some embodiments, as used herein, the term “capsule” is directed to a piece/lump of couplant which is intended for smearing beyond the margins of an electrode.

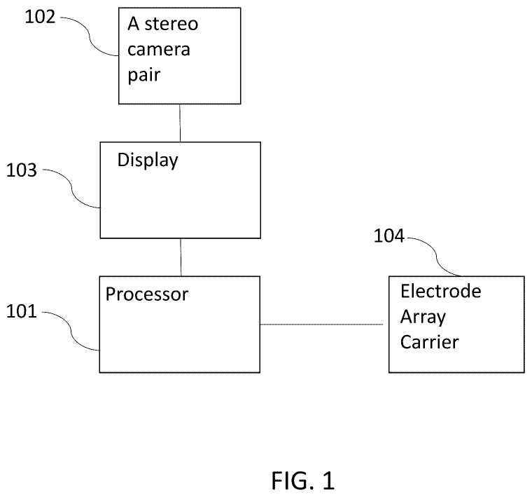

Reference is now made to FIG. 1, which schematically shows a block diagram of a system 100, for identifying a position of a plurality of electrodes in an EEG electrode array embedded to an electrode array carrier when the EEG electrode array carrier is worn on a head of a subject, according to some embodiments. System 100 includes a processor 101, a camera, which is in some embodiments, a stereo camera pair 102, a display 103 and an electrode array embedded to an electrode array carrier 104, which is connected by wires to processor 101. The electrode array carrier is worn on the head of the subject and the electrodes of the electrode array are coupled to the subject's head. Stereo camera pair 102 captures at least one image of three fiducials of the subject's head and of at least two electrodes or visible marks around electrodes placed at known positions of the EEG electrode array carrier (hereinafter visible marks) worn by the subject. Stereo camera pair 102 may be located on display 103, which presents the received EEG signals from the EEG electrode array. This configuration provides the subject immediate feedback on display 103 of what is recorded on the stereo camera pair 102. Stereo camera pair 102 may be located on any mechanical stand. The stand may have some lightning capabilities to provide more certain data to processor 101. Stereo camera pair 102, is constructed of two cameras which are synchronized and calibrated, i.e., their position and rotation are known. Each camera of stereo camera pair 102 captures the image from a different angle. The different parameters of each camera are estimated. First the distortion of the camera is estimated, and after the distortion parameter is known it is possible to undistort the image captured by this camera. Also, intrinsic parameters of the camera are estimated such as the optical center and focal length over different axis. These parameters define projection matrices which map a 3-D point onto the corresponding point in the images.

The calibration procedure consists of providing images of a chessboard which should be seen from both cameras of stereo camera pair 102. At least three chessboard images pairs have to be provided in order to calibrate the cameras. The calibration process of the cameras may be estimated only once, at the first time the subject wears the EEG electrode array carrier 104. According to some embodiments, the first time the subject wears the EEG electrode array carrier a scanning of the head is made to construct a 3D model of the head of the subject. The scanning of the head may be done in various ways, for example, a set of photographs, video, Magnetic Resonance Imaging (MRI) scanning or Computed Tomography (CT) scanning of the subject's head with or without electrodes and scanning of the subject's head with the EEG electrode array carrier. Once the 3D model of the head of the subject is constructed, it will use as a reference model, every other time the subject wears the EEG electrode array carrier, and a 3D model of the head of the subject must be reconstructed.

According to some embodiments, after the camera pair captures the at least one image of the at least three fiducials and at least two electrodes or visible marks, processor 101 executes a code which defines a coordinate system of the three fiducials and relating the at least two electrodes or visible marks around electrodes placed at known positions of the EEG electrode array carrier to the defined coordinate system. In addition, processor 101 executed a code which determines the position of each of the plurality of electrodes on the head of the subject wearing the EEG electrode array carrier. The determination of the position of each of the plurality of electrodes is based at least on the defined coordinate system and triangulation of the three fiducials and at least two electrodes or visible marks, and on a previously obtained 3D geometrical model of the subject's head and of the EEG electrode array, and a known mechanical model of the EEG electrodes' array carrier thereby, reconstructing a current 3D geometrical model of the EEG electrode array embedded to the EEG electrode array carrier worn on the subject's head.

According to some embodiments, a multi-view camera system may be used instead of stereo camera pair 102. In this case the multi-view camera system captures at least one multi view set of images at least at a single time moment, of the at least three fiducials and at least two electrodes or visible marks around electrodes placed at known positions of the EEG electrode array carrier. According to some other embodiments, a single calibrated camera may be used to capture at least one image of the at least three fiducials and at least two electrodes or visible marks. When using one calibrated camera, the determination of the position of each of the plurality of electrodes on the head of the subject wearing the EEG electrode array carrier is based on position estimation of the three fiducials and at least two electrodes or visible marks instead of triangulation thereof. According to some embodiments, the one calibrated camera or stereo camera pair or multi-view camera system may be a video camera or a still camera. According to some embodiments, the previously obtained 3D geometrical model of the subject's head and of the EEG electrode array is obtained by previously wearing by the subject the EEG electrode array carrier and measuring the geometrical model by a technician.

According to some embodiments the electrode array carrier may be a cap, a net, a carrier connecting the electrode array by springs or any type of electrode array carrier with a known mechanical model. According to some embodiments, the mechanical model of the EEG electrodes' array carrier, is un-stretchable, and preserves the geodetic distances between the electrodes. According to some other embodiments, the mechanical model of the EEG electrodes' array carrier, is stretchable, with a known value of elasticity.

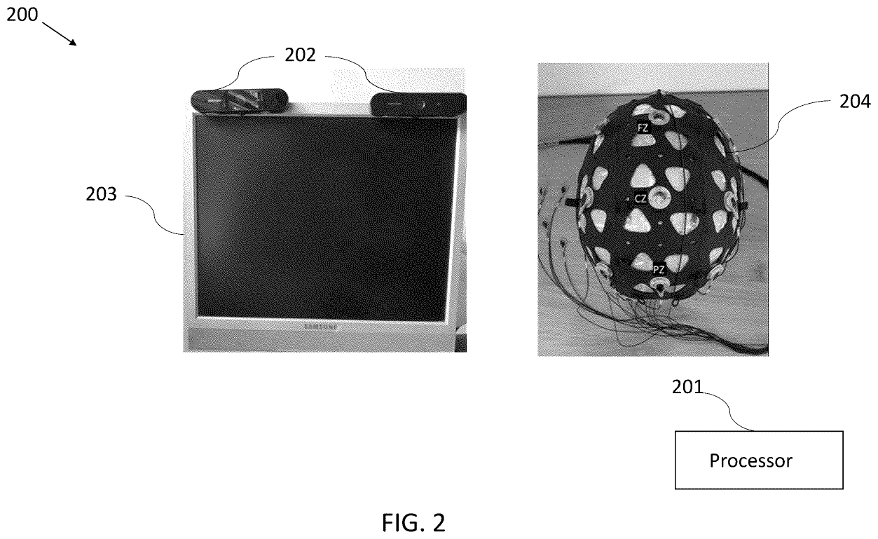

Reference is now made to FIG. 2 which schematically shows an example of a system 200 for identifying a position of a plurality of electrodes in an EEG electrode array embedded to an electrode array carrier when the EEG electrode array carrier is worn on a head of a subject, according to some embodiments. System 200 includes a stereo camera pair 202 placed on display 203, which receives signals from EEG electrode array carrier 204, for presentation on display 203. The electrode array carrier in this example is a cap. The electrode array cap 204 is connected to a processor 201. When the cap 204 is worn on the subject's head, stereo camera pair 202 captures at least one image with at least three fiducials and at least two electrodes or visible marks around electrodes placed at known positions of the EEG electrode array cap 204. Processor 201 executes a code which defines a coordinate system of the three fiducials and relates the at least two electrodes or visible marks around electrodes placed at known positions of the EEG electrode array cap 204 to the defined coordinate system. In addition, processor 201 executed a code which determines the position of each of the plurality of electrodes on the head of the subject wearing the EEG electrode array cap 204. The determination of the position of each of the plurality of electrodes is based at least on the defined coordinate system and triangulation of the three fiducials and at least two electrodes or visible marks, and on a previously obtained 3D geometrical model of the subject's head and of the EEG electrode array, and a known mechanical model of the EEG electrodes' array cap 204 thereby, reconstructing a current 3D geometrical model of the EEG electrode array embedded to the EEG electrode array cap 204 worn on the subject's head.

According to some embodiments, it is possible to successively capture one or more additional images of the head of the subject wearing the EEG electrode array carrier so that at least three elements out of the three fiducials and the at least two electrodes or visible marks, from the at least one image or from one of the multi-view set of images are visible. The at least one image or multi-view set of images are used as a reference image(s). After the additional one or more images are successively captured, the electrodes which are visible in the one or more additional images are related to the coordinate system defined in the reference image(s), and other electrodes which are visible in the one or more additional images are triangulated so that the position if the electrodes visible in the one or more additional images is identified.

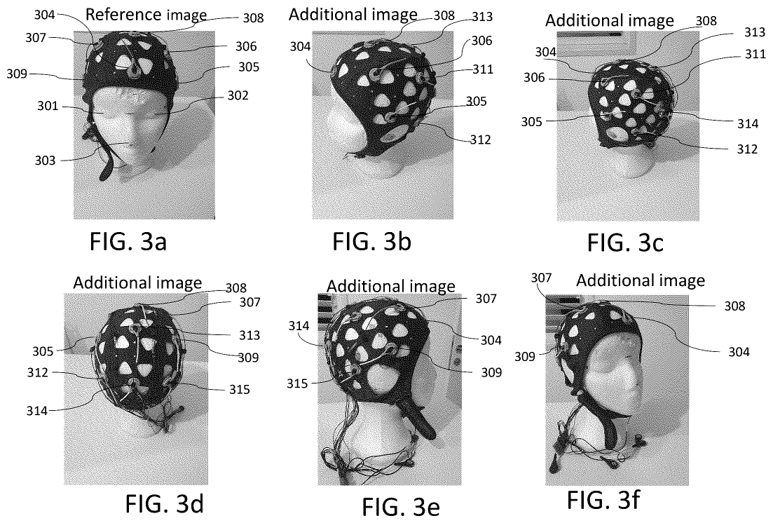

FIGS. 3a-3f schematically shows an example of one image used as a reference image (FIG. 3a) with three fiducials and two electrodes visible, and additional images successively captured (FIGS. 3b-3e) with at least three elements visible out of the three fiducials and the two electrodes, according to some embodiments. As can be seen in FIG. 3a, which uses as a reference image, three fiducials 301, 302, 303 are visible and six electrodes 304, 305, 306, 307, 308, 309 are also visible. In the successive additional images in FIGS. 3b-3f there are at least three elements (fiducials or electrodes) which are visible in the reference image and in the additional image, together with other electrodes which were not visible in the reference image of FIG. 3a. For example, electrodes 304, 305 and 308 in FIG. 3b, electrodes 304, 305, 306 and 308 in FIG. 3c, electrodes 305, 307, 308 and 309 in FIG. 3d, electrodes 304, 307 and 309 in FIG. 3e and electrodes 304, 307, 308 and 309 FIG. 3f are electrodes which are visible in the reference image of FIG. 3a and are also visible in at least one of the additional images. In this case, in FIG. 3b electrodes 304, 305, and 308 are related to the coordinate system defined in the reference image of FIG. 3a and electrodes 311, 312, 313, which are not visible in the reference image are triangulated so that the position of the electrodes 311, 312, 313 is identified.

According to some embodiments, the successive capturing of the one or more additional images of the head of the subject wearing the EEG electrode array carrier may be done when the subject's head is rotated relative to the position of the head when capturing the at least one image or when the stereo camera pair or multi-view camera system or one calibrated camera is rotated around the subject's head. Alternatively, or additionally the successive additional images may be captured by the multi-view camera system at one set of images when each camera in the multi-view camera system is positioned at a different angle so that the one set of images provides images of the head of the subject from different angels. The multi-view camera system may contain for example 3 cameras, 6 cameras, 12 cameras or the like.

In many cases, to establish stable contact of the electrode to the skin, a conductive couplant (for example, conductive gel or paste) is used and the location and the area of this couplant is the actual electrical electrode-skin contact location and area. The couplant may be spread to some distance from the electrode and, therefore, knowledge about the electrode location only (without knowledge of couplant distribution), may be insufficient to assess the electrode-skin contact location. Thus, there is a need for an additional system that can assess the couplant distribution. On the other hand, however, the couplant, usually, can move from the electrode only to relatively limited distance and therefore, knowledge about electrode locations can simplify the estimation of couplant location and distribution, so that a system for electrode localization and a system for estimating couplant distribution complement each other.

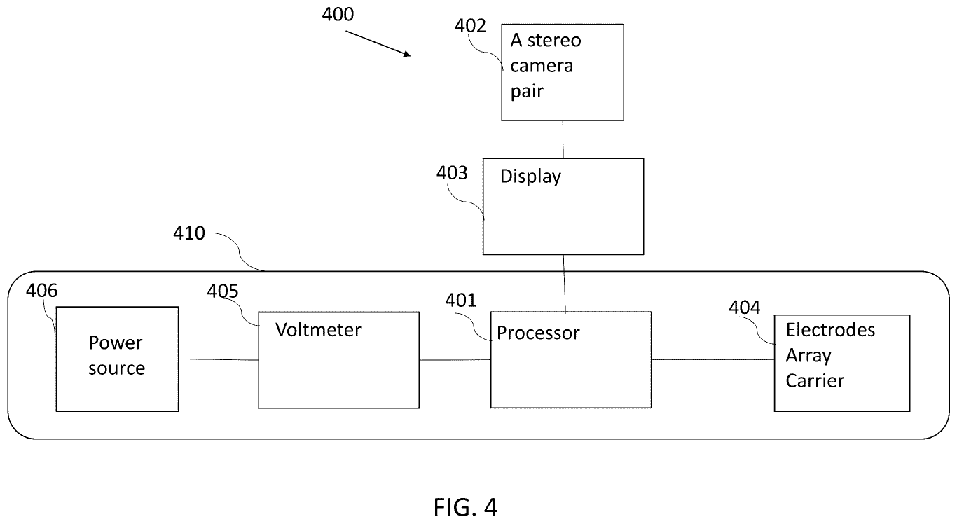

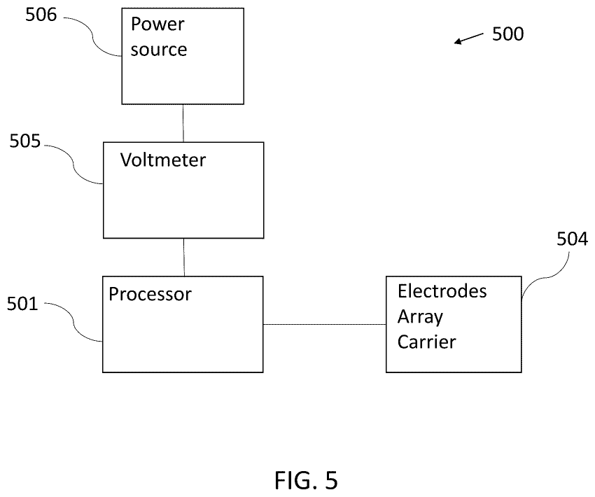

Reference is now made to FIG. 4, which depicts a schematic block diagram of an electro-optical system 400 where an electrical system 410 is connected to the optical system for identifying the position of the electrodes in the EEG electrode array embedded to an electrode array carrier when the EEG electrode array carrier is worn on a head of a subject, according to some embodiments. System 400 includes a processor 401, a stereo camera pair 402, a display 403, an electrode array carrier 404, a voltmeter 405 and an electrical power source 406. According to some embodiments, electrical system 410 is advantageously used in addition to the optical system to identify the position of the electrodes of the EEG electrode array in an accurate manner. According to some embodiments, a 3D electrical model of the EEG electrode array embedded to the EEG electrode array carrier worn on the subject's head is constructed. Electrical power source 406 injects current into one or more electrodes of the EEG electrode array, coupled to the head of the subject through the couplant. Voltmeter 405 measures the voltage response on other electrodes of the EEG electrode array, and processor 401 executes a code for constructing the 3D electrical model of the EEG electrode array embedded to the EEG electrode array carrier worn on the subject's head based on the injected currents and voltage responses measured. Processor 401 further executes a code for constructing an electrical model of the couplant spreading based at least on the voltage response as a function of the shape of the couplant spreading of all the electrodes over the head surface, and the constructed 3D electrical model of the EEG electrode array embedded to the EEG electrode array carrier worn on the subject's head. After the 3D electrical model of the EEG electrode array embedded to the EEG electrode array carrier and the electrical model of the couplant spreading are known, processor 401 executes a code for identifying the weight center of the couplant coupling the electrodes, according to the electrical model of the couplant spreading, thereby identifying the position of the electrodes. According to some embodiments, the coupling of the electrodes of the EEG electrode array to the head, may be done with dry electrodes which are coupled without couplant to the head of the subject in addition to the electrodes which are coupled with the couplant to the head of the subject. In this case, the position of the dry electrodes is identified according to a capacitive coupling function derived from the 3D electrical model of the EEG electrode array.

According to some embodiments, when constructing the electrical model of the EEG electrode array, there are certain approximations that may be made. First order approximation may be the model assuming the current mostly flows along the scalp since only a small part of the current penetrates the skull and reaches the brain. According to some embodiments, a reduction of sensitivity to the imprecision of the electrical model of the head may be achieved by mounting the electrodes, at the first time, in controlled conditions (e.g., by EEG technician), getting the electrical model of the EEG electrode array and later when reusing the system and re-wearing the electrode array carrier, using the EEG electrode array constructed at the first time as a reference. Then, when the electrodes are mounted in less controlled conditions, for example by users which are not qualified to mount the EEG electrode array using the system at a domestic environment, compare with the actual electrical model i.e., the electrical potentials measured on electrodes is compared to the reference model measurement of the first time.

According to some embodiments, after the electrical model of the EEG electrode array is constructed the electrical model of the couplant spreading can be constructed. According to some embodiments, if the electrical couplant is not spread properly filling the gap between the electrode and the scalp of the subject (properly spread couplant is mostly filling the volume between the electrode and the scalp) it causes short-circuit areas of the scalp and hence influence the electrical model of the head. The spreading of the couplant model can be parameterized by assuming areas with smooth perimeter of perfectly conducting couplant around the electrodes, sampled in about 7 points along the azimuthal coordinate and spline interpolated. To estimate and construct the spreading of the couplant model and identify bridges, the current may be induced in each of the electrodes (similar to current injection for contact impedance measurements) and the voltage relative to the reference electrode can be measured. For example: for 16 electrodes in the EEG electrode array, current is injected to one electrode and voltage is induced in the other 15 electrodes, relative to the reference electrode (the electrode to which current was injected), this procedure repeats 16 times—for each of the 16 electrodes. However, it can be proven that the impedance matrix is symmetrical. Hence, there are (16*15)/2 independent measurements. The (16*15)/2=120 independent measurements can be performed estimating the parameters of the coupling areas.

After the spreading of the couplant model is constructed, it is easy to detect cases where the couplant is not properly filling the gap between the electrode and the scalp of the subject. A particular case of a shape change may be a distance change. For example, when the couplant corresponding to two different electrodes is much closer than the distance between the two corresponding electrodes, the voltage differences from the current injected from another electrode drops significantly in comparison to the nominal reference value of the constructed model. In addition, in case of electrical bridges (i.e., shortcut) as a special case of couplant from different electrodes coming very close or touching each other, may also be easily detected as the voltage differences between the short-circuited electrodes is negligible. Advantageously, the combined electro-optical system provides both the location of the electrodes and the estimation of the spreading of the couplant enabling to analyze properly the recorded EEG signals.

The above-mentioned problem may be mathematically formulated as a problem of finding the contact areas:

Assuming the centers of the electrodes 1, . . . , L are located at positions r₁, . . . , r_{L} along the scalp. U₁, . . . , U_{L} denotes the areas on the scalp where the electrical couplant of the corresponding electrodes contacts with the skin. The corresponding set of parameters αᵢⱼ can be denote by the matrix A:

$$A = \{ {α}_{ij} \vert i = 1 , .. , L ; j = 1 , .. , M \}$$

Each column of this matrix contains the parameters defining the contact area of the corresponding electrode. A z-function Z (r, r′, A) is also defined, which by definition is the voltage measured at point r′ when the unit electrical current is injected into point r and withdrawn from the reference electrode. Clearly, this function is dependent both on the electrical conductivity distribution within the head σ(r) and the contact areas of the couplant.

The discretization of this function if measured only on the electrodes is called the z-matrix:

$$Z = \{ Z ( r , {r}^{′} , A ) \vert r = {r}_{1} , … , {r}_{L} ; {r}^{′} = {r}_{1} , … , {r}_{L} \}$$

In Electrical Impedance Tomography (EIT) the conductivity distribution within the region enclosed by the electrodes is estimated from the z-matrix measurements. It is noted here that the perfectly conducting couplant introduces singularity in the classic EIT problem. Hence, the electrical conductivity of the couplant should be considered finite (possibly high).

It is assumed in the calibration step that the head model was first estimated from the EIT measurements when the contact areas were known and defined by the parameters Ao.

Given the estimated head model, the z-matrix can be calculated for any set of locations. Suppose the contact areas of the couplant are altered corresponding to an unknown set of parameters A. Now the conductivity distribution which was estimated at the calibration step can be analyzed, and just assume the unknown are the parameters of the contact areas. The optimal parameters of the contact areas Aₒₚₜ can be found as the solution to the optimization problem minimizing the discrepancy (for instance Frobenius norm) between the measured z-matrix Zₘ and the calculated z-matrix Z_{calc} in the assumption of certain set of the parameters defining the contact areas:

$${A}_{opt} = arg min {‖ {Z}_{m} - {Z}_{calc} ‖}_{2}$$

Since the solution can be not unique, the expression may be regularized for example by requiring the minimal overall contact area.

According to some embodiments, there are provided capsules with a constant and known amount of couplant, which may be embedded to the EEG electrode array carrier, to provide a limited distribution of the couplant, and to eliminate a wrong use of the couplant by using a large amount of couplant with very high distribution. The capsule may be opened for example with pressure. Optionally, the capsules may be placed in proximity to the electrodes in the EEG electrode array carrier, or alternatively, each electrode of the EEG electrode array may be placed inside the capsule, and when external pressure is applied, couplant is moved to the space between the electrode and the head (i.e., scalp) of the subject. According to some embodiments, to optimize the EEG electrode array, such electrode-capsules may be combined with electrode-stickers. The electrode-capsules are applied to the part of the head with hair, while electrode-stickers are applied to the part of the head without hair, for example the forehead. The electrode-skin contact of electrode-sticker is better defined by the optical system only, since glue, which is part of the sticker, around the electrode, prevents couplant dispersion. Therefore, inclusion of electrode-stickers to the electrical model can increase the accuracy of couplant dispersion in electrode-capsules. In addition, electrode-stickers can serve as mechanical anchors for the whole EEG electrode array (electrode-capsules and electrode-stickers).

According to some embodiments, electrical system 410 may be independent of the optical system and may be used as an independent system without the optical system for identifying the position of electrodes in an EEG electrode array embedded into an EEG electrode array carrier, worn by a subject. For example, as shown in FIG. 5 which schematically shows an electrical system 500 for identifying the position of electrodes in an EEG electrode array embedded into an EEG electrode array carrier, worn by a subject, according to some embodiments. System 500 includes a processor 501, an electrode array carrier 504, a voltmeter 505 and an electrical power source 506. According to some embodiments, a 3D electrical model of the EEG electrode array embedded to the EEG electrode array carrier worn on the subject's head is constructed. Electrical power source 406 injects current into one or more electrodes of the EEG electrode array, coupled to the head of the subject through the couplant. Voltmeter 505 measures the voltage response on other electrodes of the EEG electrode array, and processor 501 executes a code for constructing the 3D electrical model of the EEG electrode array embedded to the EEG electrode array carrier worn on the subject's head based on the injected currents and voltage responses measured. Processor 501 further executes a code for constructing an electrical model of the couplant spreading based at least on the voltage response as a function of the distance between the couplant of each of the measured electrodes, and the constructed 3D electrical model of the EEG electrode array embedded to the EEG electrode array carrier worn on the subject's head. After the 3D electrical model of the EEG electrode array embedded to the EEG electrode array carrier and the electrical model of the couplant spreading are known, processor 501 executes a code for identifying the weight center of the couplant coupling the electrodes, according to the electrical model of the couplant spreading, thereby identifying the position of the electrodes.

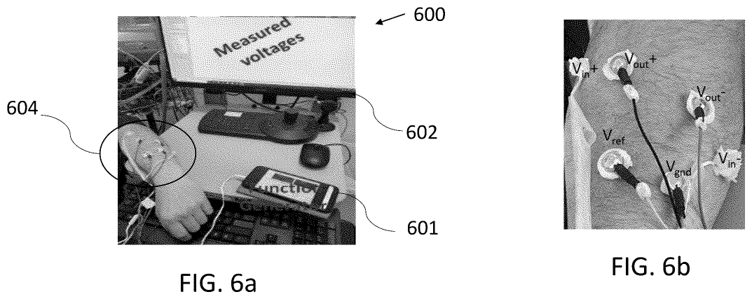

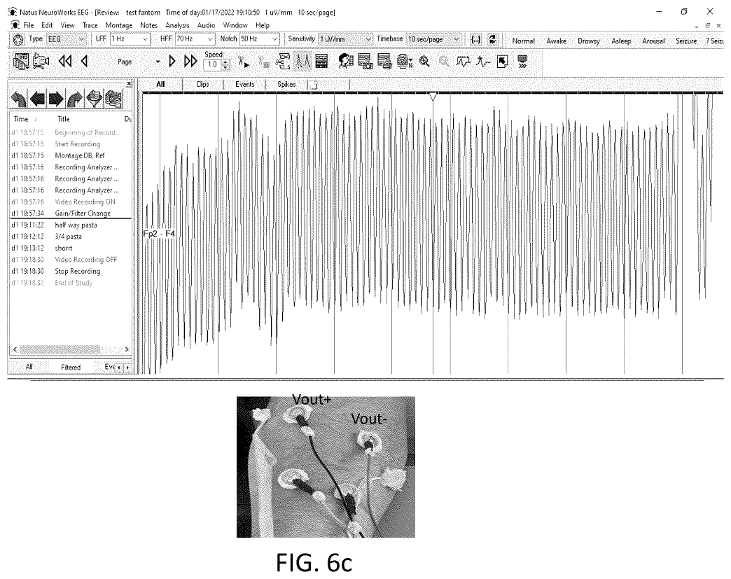

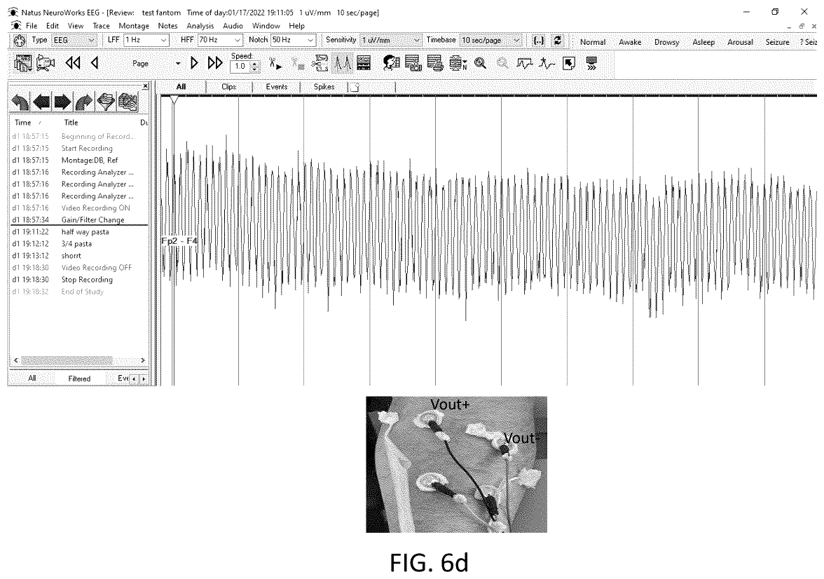

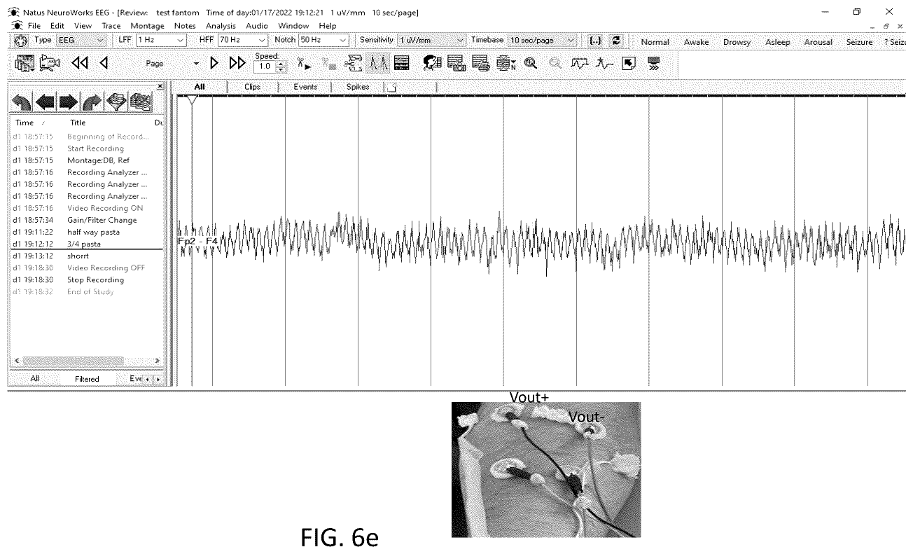

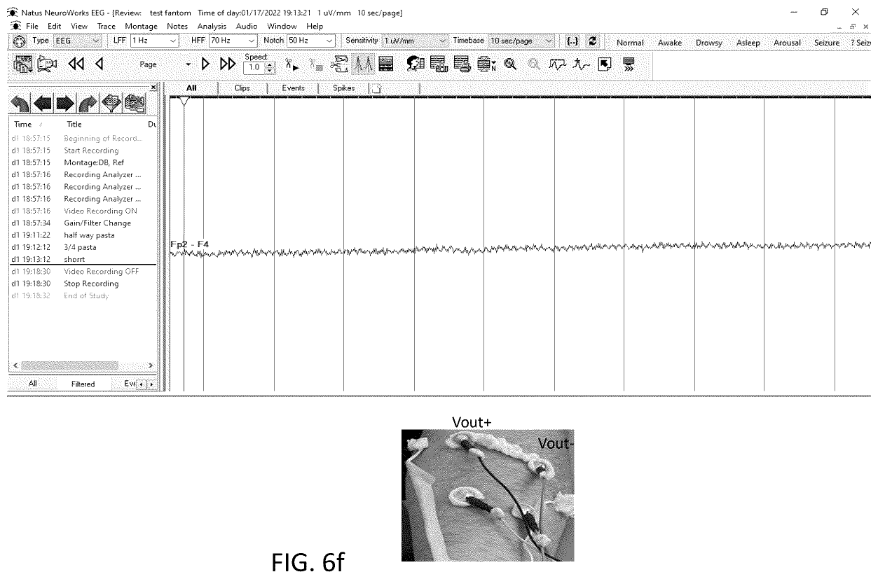

Reference is now made to FIGS. 6a-6f which schematically show an example for an electrical system according to some embodiments and results of exemplary cases of proper and improper spreading of the couplant coupling the electrodes in the EEG electrode array to a skin of a subject, according to some embodiments. FIG. 6a shows system 600, with smartphone 601 which is used as an electrical power source and as a processor, EEG electrode array 604 coupled to the forearm skin of a subject with couplant which is a paste, and a display 602 for presenting the signals received from EEG electrode array 604. Smartphone 601 using as an electrical power source and with a function generator base on the analog output of the smartphone 601 injects current to electrodes in EEG electrode array 604. The voltage measurements were performed by a voltmeter (not shown) of an EEG system. The current frequency was set to 10 Hz and measurements for different conductive paste spread were performed. FIG. 6b schematically shows EEG electrode array 604, which includes Vin+, Vin− electrodes which are the injecting electrodes to which current is injected. Vout+, Vout− electrodes which are the EEG electrodes where the voltage is measured. Vref is the reference electrode and Vgnd is the ground of the EEG system. FIG. 6c schematically shows an example where the paste is properly spread and is filling the gap between the electrodes and the skin of the subject. In this case the result shown on the display shows voltage difference that corresponds to the couplant spreading model. FIG. 6d schematically shows an example where the paste is not properly filling the gap between the electrodes and the skin of the subject as the paste spreads out with a low spread of the Vout− electrode. In this case, the result of the measured voltage difference between the Vout− and Vout+ electrodes drops, and it is lower than the voltage of the couplant spreading model. In FIG. 6e a schematic example of a case where the paste is not properly filling the gap between the electrodes and the skin of the subject is shown. In this case the paste spreads out with a high spread of the Vout− electrode. The result of the voltage difference measurement in this case is even lower than the voltage difference measured in FIG. 6d. In FIG. 6f a schematic example of a case of a full bridge is shown, where the paste of the Vout− electrode spreads until it connects to the paste of the Vout+ electrode. In this case the Vout− and Vout+ electrodes are short-circuited, and so the voltage difference measured between the two Vout− and Vout+ electrodes is negligible.

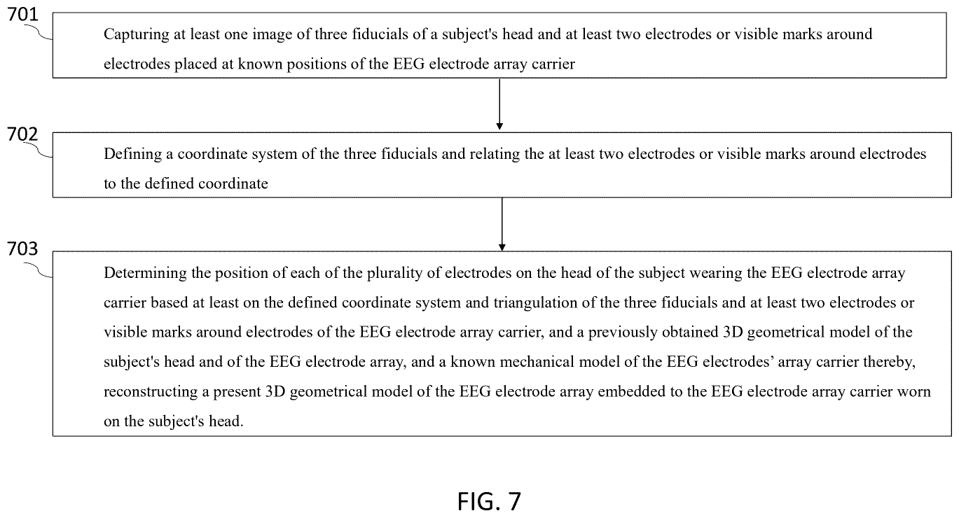

Reference is now to FIG. 7, which schematically shows a flow chart of a method for identifying a position of a plurality of electrodes in an EEG electrode array embedded to an electrode array carrier when the EEG electrode array carrier is worn on a head of a subject, according to some embodiments. At 701, at least one image of three fiducials of a subject's head and at least two electrodes or visible marks around electrodes placed at known positions of the EEG electrode array carrier is captured by a stereo camera pair. At 702, a coordinate system of the three fiducials is defined, and the at least two electrodes or visible marks are related to the defined coordinate system of the three fiducials, this process is executed by a processor which executed a code for processing the data in the at least one image captured by the stereo camera pair. At 703, the position of each of the plurality of electrodes on the head of the subject wearing the EEG electrode array carrier is determined based at least on the defined coordinate system and on triangulation of the three fiducials and at least two electrodes or visible marks, and on a previously obtained 3D geometrical model of the subject's head and of the EEG electrode array, and a known mechanical model of the EEG electrodes' array carrier, thereby reconstructing a present 3D geometrical model of the EEG electrode array embedded to the EEG electrode array carrier worn on the subject's head.

According to some embodiments, when a stereo camera pair is used, the triangulation of the three fiducials and at least two electrodes or visible marks is used to find the respective position of the three fiducials and the at least two electrodes and visible marks at other angles which were not captured by the stereo camera pair according to the previously obtained 3D geometrical model of the subject's head and of the EEG electrode array. Then, the position of the rest of the electrodes which are not visible in the at least one image captured by the stereo camera pair is also identified. According to some embodiments, a multi-view camera system comprised of a plurality of cameras may be used instead of the stereo camera pair. In this case the multi-view camera system captures a set of multi-view images of the head of the subject at one time. Alternatively, a single calibrated camera may be used, capturing one image of the head of the subject. In this case, instead of triangulation of the three fiducials and at least two electrodes or visible marks, a position estimation process is performed to identify the position of the three fiducials and at least two electrodes or visible marks in different angles based on the previously obtained 3D geometrical model. Then, the position of the rest of the electrodes which are not visible in the one image captured by the one calibrated camera are also identified. When the single calibrated camera is used, it is determined on each image of the head with the EEG electrode array carrier, which part of the head surface the image represents, based on the previously obtained 3d model of the subject's head. For example, based on the head landmarks detection (e.g., nose, eyes, ears and the like) the part of the head surface represented in the image is determined. The camera position relative to the head is also determined (for instance by at least 3 fiducials or other known landmarks on the head surface). Based on the known head surface and on the known calibrated camera position, the part of the head which is occluded by the EEG electrode array carrier may also be determined on the image. When the carrier of the EEG electrode array is with a known mechanical model (elasticity and the distances between the electrodes) it can be assumed that it conforms to the head surface. Hence the electrodes of the EEG electrode array on the carrier can be related to the facial coordinate system by comparing the part of the image occluded by the EEG electrode array carrier with the head surface projected to the image having the known position and orientation of the camera.

Advantageously, the use of a stereo camera pair capturing one image greatly lower the cost of the system as it requires only two cameras, it is easy and simple to use, yet it provides stable and accurate results.

According to some embodiments, additional steps may be carried out to provide even more stable and accurate results by successively capturing one or more additional images of the head of the subject wearing the EEG electrode array carrier.

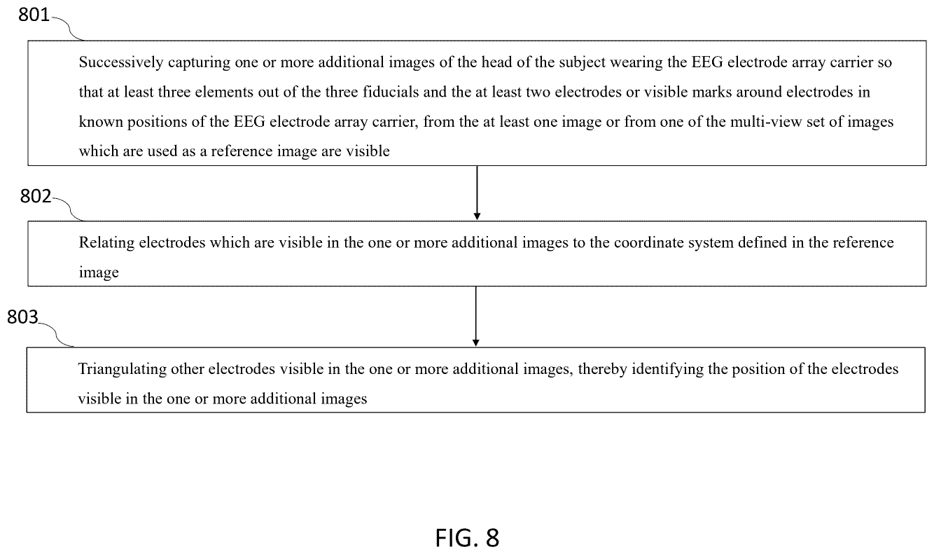

Reference is now made to FIG. 8, which schematically shows a flow chart of a method for identifying a position of a plurality of electrodes in an EEG electrode array embedded to an electrode array carrier when the EEG electrode array carrier is worn on a head of a subject, by successively capturing additional images of the head of the subject wearing the EEG electrode array carrier, according to some embodiments. At 801, one or more additional images of the head of the subject wearing the EEG electrode array carrier are successively captured so that at least three elements out of the three fiducials and the at least two electrodes or visible marks from the at least one image or from one of the multi-view set of images which are used as a reference image are visible. At 802, electrodes which are visible in the one or more additional images are related to the coordinate system defined in the reference image. At 803, other electrodes visible in the one or more additional images, which were not visible in the reference image(s) are triangulated and thereby, the position of these electrodes which are visible in the one or more additional images but were not visible in the reference image(s) is identified.

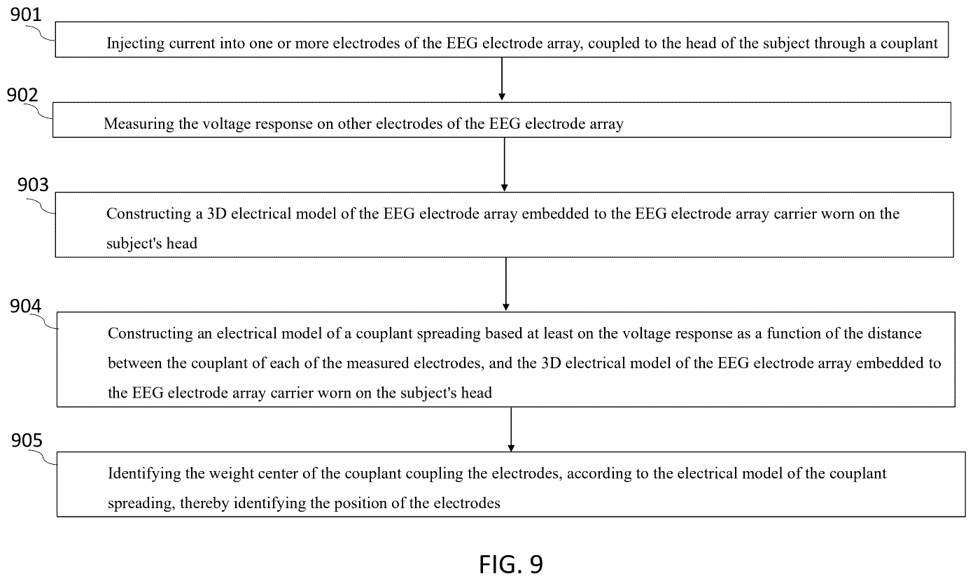

Reference is now made to FIG. 9, which schematically shows a flow chart of an electrical method for identifying a position of a plurality of electrodes in an EEG electrode array embedded to an electrode array carrier when the EEG electrode array carrier is worn on a head of a subject, according to some embodiments. At 901, current is injected into one or more electrodes of the EEG electrode array, coupled to the head of the subject through a couplant. At 902 the voltage response is measured on other electrodes of the EEG electrode array (i.e., not the electrodes to which current was injected to). At 903, a 3D electrical model of the EEG electrode array embedded to the EEG electrode array carrier, coupled to the head of the subject through a couplant is constructed. At 904 an electrical model of a couplant spreading is constructed, based at least on the voltage response as a function of the shape of the couplant spreading of all the electrodes of the EEG electrode array over the subject's head surface, and on the 3D electrical model of the EEG electrode array embedded to the EEG electrode array carrier worn on the subject's head. Optionally, the spreading of the couplant may be measured through electrodes which are not initially coupled to the subject's head, but due to the spreading of the couplant the unconnected electrodes got connected to couplant. At 905, the weight center of the couplant coupling the electrodes, is identified, according to the electrical model of the couplant spreading, thereby identifying the position of the electrodes. According to some embodiments, the electrical method may be used in addition to the optical method, advantageously providing more accurate results and position identification of the electrodes in the EEG electrode array than the optical method. When using the electro-optical method, the electrical models of the EEG electrode array and of the couplant spreading are used as a reference for the reconstructed 3D geometrical model of the EEG electrode array, thereby constructing a 3D electro-geometrical model of the EEG electrode array embedded to the EEG electrode array carrier worn on the subject's head.

According to some embodiments, the coupling of the electrodes of the EEG electrode array to the head may be done with dry electrodes which are coupled without couplant to the head of the subject in addition to the electrodes which are coupled with the couplant to the head of the subject. In this case, the position of the dry electrodes is identified according to a capacitive coupling function derived from the 3D electrical model of the EEG electrode array worn on the subject's head.

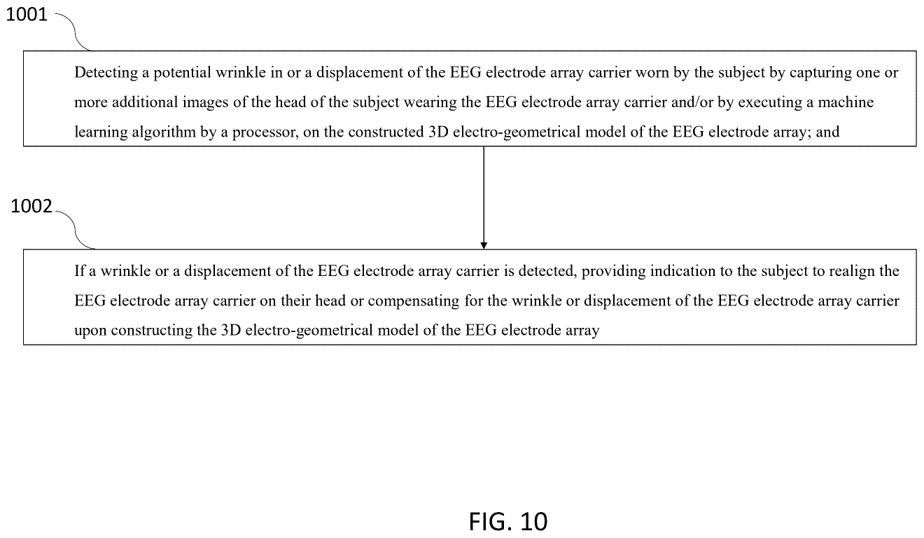

Reference is now made to FIG. 10, which schematically shows a flow chart of an electro-optical method for identifying a position of a plurality of electrodes in an EEG electrode array embedded to an electrode array carrier when the EEG electrode array carrier is worn on a head of a subject, when a wrinkle is detected in the electrode array carrier or a displacement of the EEG electrode array carrier is detected, according to some embodiments. The presented steps may be performed in addition to steps 701 to 703 and 901 to 905. At 1001, a potential wrinkle or a displacement is detected in the EEG electrode array carrier worn by the subject by capturing one or more additional images of the head of the subject wearing the EEG electrode array carrier. Alternatively, or additionally, the potential wrinkle or displacement of the EEG electrode array carrier may be detected by executing a machine learning algorithm by a processor, on the constructed 3D electro-geometrical model of the EEG electrode array. At 1002, if a wrinkle or a displacement of the EEG electrode array carrier is detected, an indication is provided to the subject to realign the EEG electrode array carrier on the head, for example by a message presented on the display, or by a voice indication or the like. Another option may be compensating for the wrinkle or displacement of the EEG electrode array carrier upon constructing the 3D electro-geometrical model of the EEG electrode array, thus obviating the need to provide an indication to the user to realign the EEG electrode array carrier on the head. In this case when a wrinkle or a displacement is detected the 3D electro-geometrical model is reconstructed taking the wrinkle or displacement of the EEG electrode array into consideration and compensating for it. According to some embodiments, the machine learning algorithm may be a deep neural network trained on labeled database records of wrinkles or displacements of the EEG electrode array carrier. The database records of wrinkles or displacements of the EEG electrode array carrier may be optical records, electrical records, or both.

According to some embodiments the electrical method for identifying a position of a plurality of electrodes in an EEG electrode array embedded to an electrode array carrier when the EEG electrode array carrier is worn on a head of a subject, may be used independently from the optical method, and thus it may be used as a sole method for identifying the position of the electrodes in an EEG electrode array embedded to an EEG electrode array carrier worn on a subject's head.

### EXAMPLES

### Example 1—Optical Experimental System

To tune the parameters of the method described above, for example in FIGS. 7-8, an example for an experimental system was built. The parameters taken into account included for instance the number of cameras, resolution of these cameras, the optimal locations of the cameras, how many rotations the patient should turn their head, the resulting precision of the electrodes' locations, and the like. Based on these considerations several design requirements for the system were set:
1) Flexibility to put cameras in different locations;
2) High resolution (which can be controlled), high sensitivity of the cameras;
3) Ability to rotate the part of the system on which the cameras are mounted;
4) Dimensions of the system (sufficiently large to enable the head to be captured fully from different sides, but not too large to prevent bulkiness and enable high resolution and rigidness of the structure);
5) Connectivity requirements to enable video data streaming/saving from multiple cameras
6) Synchronization of the cameras.

Further requirements of the study included privacy of the patient and other requirements such as weight, cost effectiveness, rigidness and the like.

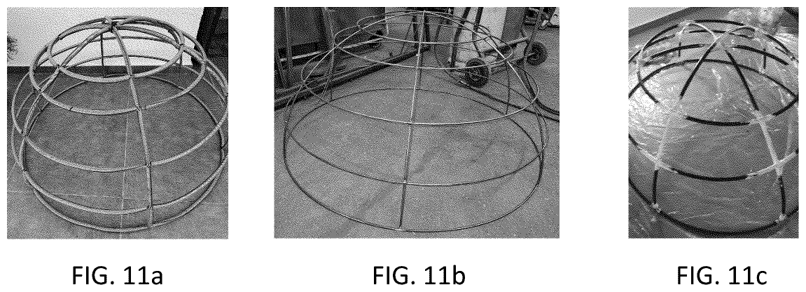

After considering several options for the part of the system on which the cameras are mounted on, a hemispherical structure was chosen as shown in FIGS. 11a-11c, which enables placing cameras at different positions over the surface of the hemisphere.

A first prototype of a structure shown in FIG. 11a was built from plastic tubes. However, while suitable for the initial experiments and cost effective, this solution was abandoned since over time it started to change its shape under the weight of the cameras. A second prototype was based on a metal construction as can be seen in FIG. 11b. To prevent possible hitting of the construction on the head of the patient, resin encapsulation was applied around the metallic structure and the final prototype of the structure is shown in FIG. 11c.

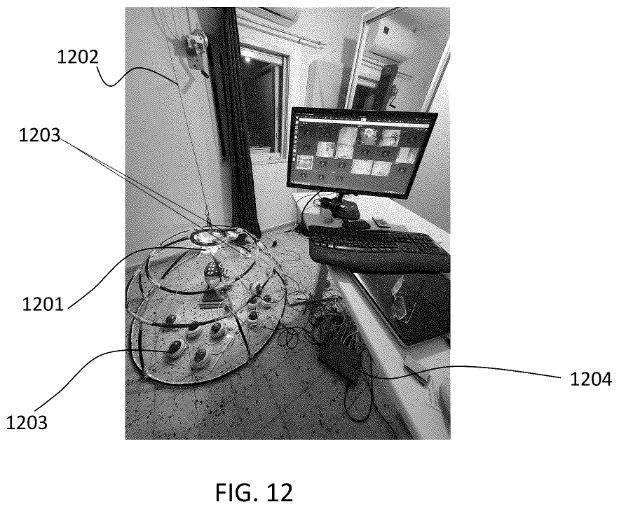

FIG. 12 schematically shows an example of a final prototype of an optical system where the final prototype of the structure of FIG. 11 is connected to a processor and a screen, according to some embodiments. The prototype structure includes a light source 1201, a lifting mechanism 1202, which enabled to control the height of the construction above the head of the patient. Using the lifting mechanism, the structure can remain under the ceiling while not in use to reduce the area requirements for the equipment. The structure enables rotation. Different fixation mechanisms were proposed and tested, including fixating by the hand of the technician and clips mechanism to fixate the structure to the sitting chair. The leading option is fixating the structure to the sitting chair, however other options are still tested.

After considering several options for cameras, Internet Protocol (IP) Gadinan 5Mp security cameras such as camera 1203 powered via a power switch 1204 were chosen. This solution is cost effective, enables connecting high resolution cameras and enables connecting the IP cameras without connecting to the global internet network, but rather connecting to the local network which can be established using a PC computer, a router and the power switch. A power-over-ethernet technology enables reducing the number of cables by providing both the power and the communication over the same cable.

A Python code which acquires the videos from the IP cameras through the ethernet and saves the files to the PC computer was developed. A synchronization of about one second was achieved.

### Example 2—Phantom Experiment for Conductive Paste Smearing Assessment

An experiment was performed in the EEG laboratory of the Hadassah University Medical Center—Ein Karem.

The goal of the experiment was to demonstrate the ability to assess the conductive paste distribution along the scalp using externally injected electrical current.

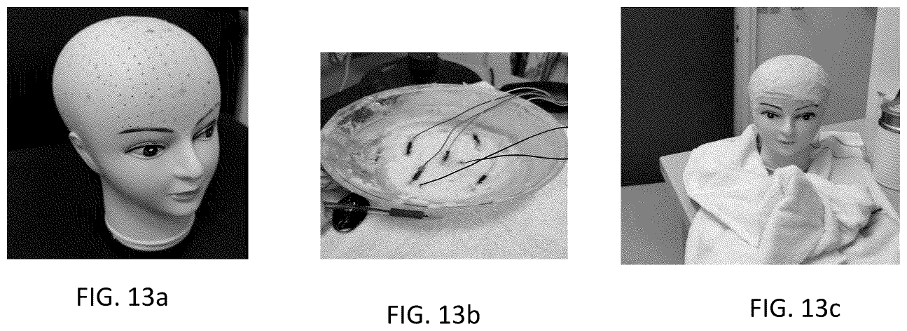

The phantom was manufactured utilizing plastic head moulage. Openings over the phantom head with a diameter of about 1 mm were created by drilling where the distance between each of the openings is of about 1 cm. FIG. 13a shows a phantom head with the openings, according to some embodiments.

A soft conductive material (SCM) was prepared by mixing soft white paraffin with conductive paste (“Ten20®”). The optimal proportion of these two ingredients in the SCM was achieved iteratively. First, the soft white paraffin and “Ten20®” were mixed in the proportion of 10:1 respectively. The EEG electrodes, including reference, ground and several leads were connected to a plate containing the mixed material as can be seen at FIG. 13b and the electrodes were connected to an EEG device (“Natus®”, not shown). The baseline recording of the received signals was noisy. Next, the proportion of the conductive paste was gradually increased up to a proportion of 3:10 (3-conductive paste and 10-parafin), when the baseline recording appeared without substantial noise.

The SCM was inserted inside the head moulage and smeared on outside the head moulage, simulating brain and scalp respectively. Thus, an electrical connection was established between the outer part and the inner part of the phantom by soft conductive material (SCM) bridges through the multiple openings in the phantom. FIG. 13c shows the head moulage smeared with the SCM, according to some embodiments.

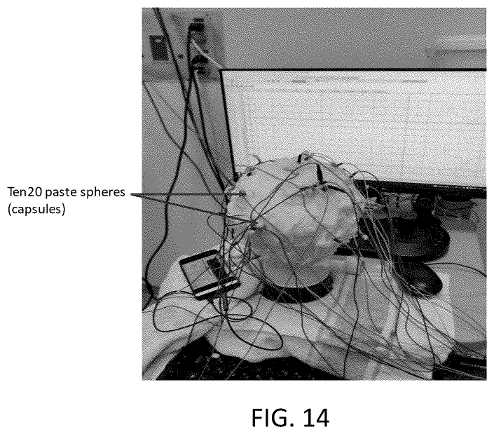

Capsules (spheres) of conductive paste were placed on the back part of the phantom and EEG electrodes (ground, reference, and other electrodes) were inserted into these capsules (spheres) and were connected to the EEG device as can be seen in FIG. 14.

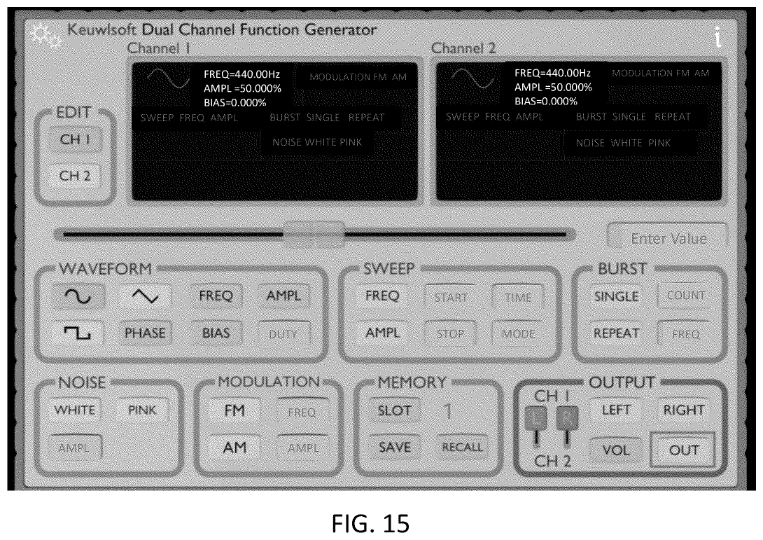

A sinusoidal electrical current was generated by the audio output of a mobile phone using Function Generator application. FIG. 15 shows the user interface screen of the Function Generator application, according to some embodiments. The frequency of the sinusoidal current was set to about 10 Hz. The distribution of sinusoidal changes of electrical field were observed between the electrodes in virtual longitudinal bipolar montage.

To generate sinusoidal variations of electrical field two cables were connected to a mobile phone output from one end, and to two conductive paste capsules (spheres) on the surface of the phantom from a second end.

During the application of sinusoidal current the conductive paste in the capsules was smeared to about half of distance between the capsules associated with some electrode pairs and the sinusoidal amplitudes before and after conductive paste smearing were observed in virtually bipolar channels related to these electrode pairs.

Current was injected between electrodes Fp2 and C4 and the voltage response was measured on electrodes T4 and T6.

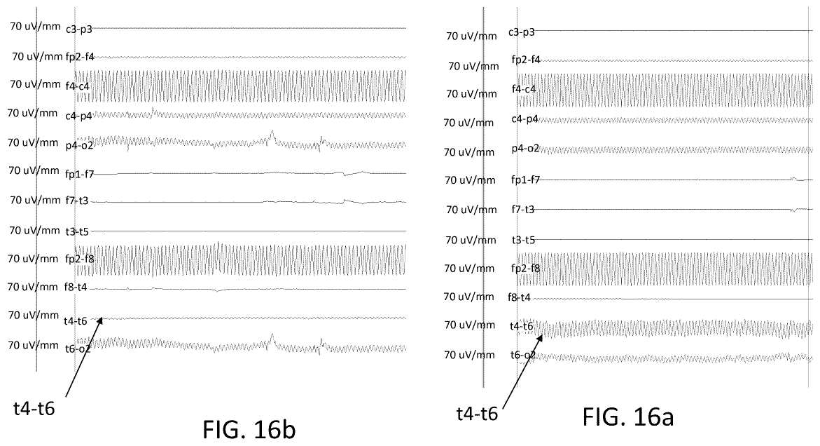

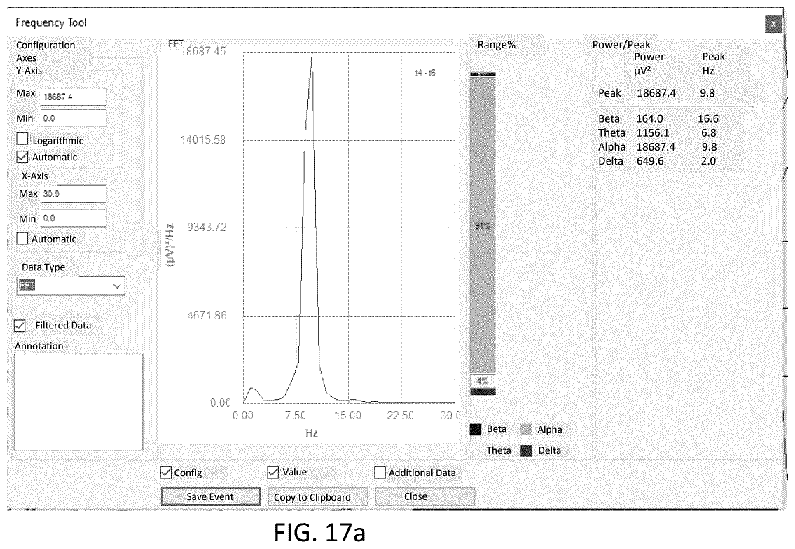

FIG. 16a shows the results of the voltage measurements between electrodes T4 and T6 before paste smearing, according to some embodiments. FIG. 16b shows the results of the voltage measurements between electrodes T4 and T6 after paste smearing according to some embodiments. It can be seen that the amplitude after the paste smearing is lower (to understand how much lower, a furrier transform of channel T4-T6 was conducted and described in FIGS. 17a-17b). This is due to the fact that after paste smearing the gradient (and resistance) between the edges of the paste of each electrode (T4 and T6) is lower.

FIG. 17a shows the result of a furrier transform of channel T4-T6 before paste smearing, according to some embodiments. FIG. 17b shows the result of a furrier transform of channel T4-T6 after paste smearing, according to some embodiments.

After the conductive paste is smeared to about half of the distance between electrodes T4 and T6, a power peak at 9.8 Hz sinusoidal trace was reduced in more than hundredfold: from 18687 to 168 μV²/Hz. By analyzing the voltage response, the distances between the edges of the paste of the electrodes can be estimated, thereby, the conductive paste distribution along the scalp using externally injected electrical current was assessed.

### Example 3—Electrode Array

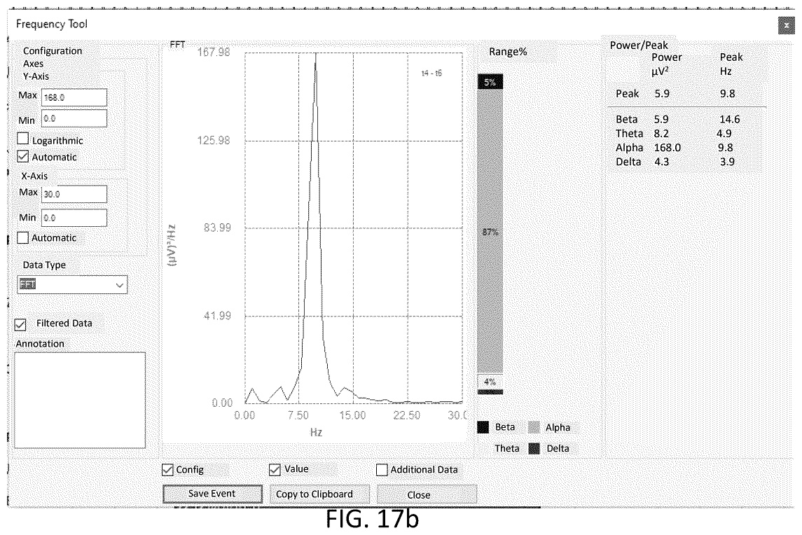

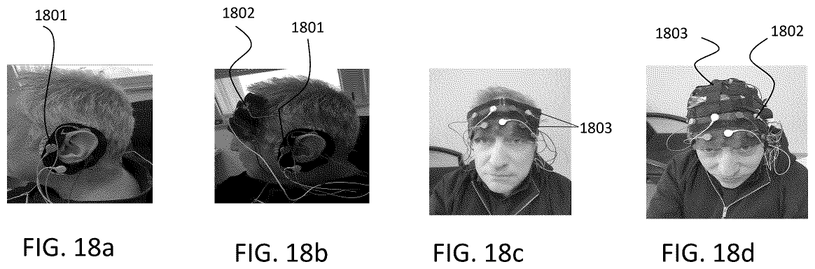

The electrode array of the system disclosed herein, according to some embodiments, is composed of two types of electrodes: (i) sticker-electrodes and (ii) capsule-electrodes. Sticker-electrodes are arranged in two sets: periauricular and frontal. Capsule-electrodes are arranged in a cap. The cap is fixed to attachment band/s of the frontal sticker-electrode set. FIG. 18a shows an example of a periauricular sticker-electrodes attached to the head of a subject, such as electrode 1801, according to some embodiments. FIG. 18b shows an example of periauricular sticker-electrodes and a side view of frontal sticker-electrodes (such as electrode 1802) attached to the head of a subject, according to some embodiments. FIG. 18c shows an example of a front view of frontal sticker-electrodes attached to the head of a subject, according to some embodiments. In this frontal view, the periauricular sticker-electrodes, which are also attached, cannot be seen. FIG. 18d shows an example of a combination of frontal sticker-electrode set and a capsule-electrode cap 1803 attached to the head of a subject, according to some embodiments. The periauricular sticker-electrodes, are also attached however they cannot be seen.

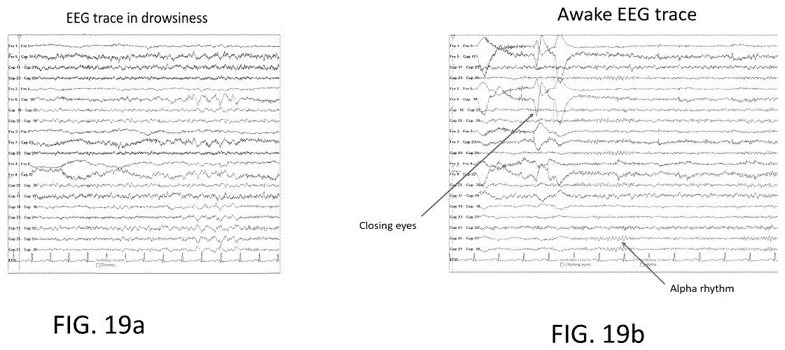

EEG electrodes with broad contacts provide more stable signals. This is achieved since signals with high spatial frequencies are filtered out by spatial averaging in the site of the contact. The EEG electrodes with broad contacts filter out signals that can include local skin potentials, and local muscles signal related to scalp muscles. On the other hand, the signals from the depth of sulci, from distant sources or fields related to large circuits (the most epileptic sources belong to this category) are not suppressed. Moreover, the SNR is improved for these signals. Therefore, electrodes with broad based contacts mostly emphasize epileptic signals, suppressing both extracranial electric signals (skin and muscle) and background brain noise. FIGS. 19a-19b schematically show EEG traces during drowsiness and wakefulness respectively, using the capsule electrodes with broad contacts, according to some embodiments. It can be seen that the EEG measurements using the capsule electrodes provide results in the same standard of traditional EEG measurements using standard electrodes, including for example the recognition of closed eyes point and alpha rhythm.

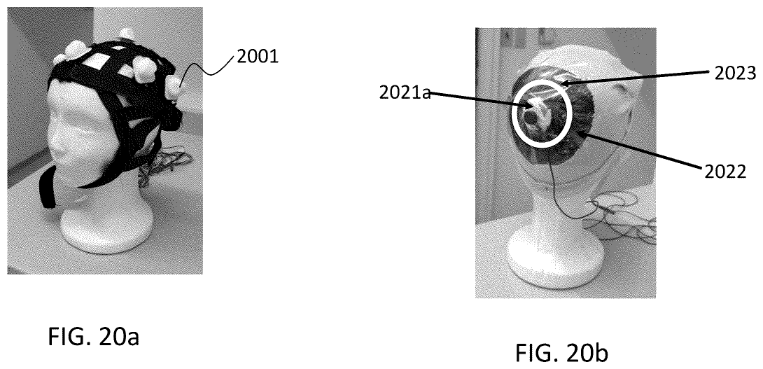

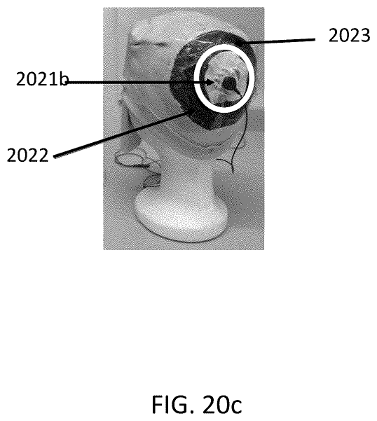

The conductive paste capsules are smeared accepting thin disk-like shape. FIG. 20a shows an example of conductive paste capsules 2001 smeared as thin disk-like shapes, according to some embodiments. If the diameter of a capsule is 1.5 cm and the capsule is smeared to the thickness of 2 mm, then the surface of contact is 9 cm² and the radius of the disk is 1.7 cm. In such cases the distance between neighboring contact margins is as described in Table 1:

TABLE 1

| Distance between electrode centers | Distance between contact margins |
|---|---|
| 5 cm | 1.6 cm |
| 6 cm | 2.6 cm |
| 7 cm | 3.6 cm |
| 8 cm | 4.6 cm |

According to some embodiments, “couplant spreading” or “couplant smearing” may be understood as a pattern/design or area/volume/region, and the edges/borders of this area/volume are used to estimate the distances between the electrodes contacts.

According to some embodiments, to visualize the capsule smearing, the EEG cap should be transparent, or at least to include transparent windows with marked borders of preferable smearing. An additional role of these transparent windows in the cap is to enable the photogrammetry of margins of smearing capsules. Thus, the intercontact distances are measured both optically and electrically (by injecting electrical currents into different electrode pairs as described in example 2 above). Using the electrical model, the relation between amplitude and intercontact distance can be assessed using principles described in Epstein, Charles M., and Gail P. Brickley. “Interelectrode distance and amplitude of the scalp EEG.” Electroencephalography and clinical neurophysiology 60, no. 4 (1985): 287-292.

According to some embodiments, when using the capsule electrodes, a transparent cap or a cap with transparent window(s) may be used, thereby enabling the camera(s) of the optical model of the system to capture images of the paste smeared on the head of the subject and identify the conductive paste edges, using image processing algorithms. In addition, a border may be marked on the transparent cap or window to indicate to the user a recommended borders for the paste smearing.

FIG. 20b shows an example of a transparent window in an EEG cap and capsule electrode before paste smearing, according to some embodiments. Capsule electrode 2021a is a capsule electrode before paste smearing. Window 2022 is a transparent window in the EEG cap and border 2023 is marked for recommended paste smearing. FIG. 20c shows an example of a transparent window in an EEG cap and capsule electrode after paste smearing, according to some embodiments. Capsule electrode 2021b is the capsule electrode after paste smearing. The paste was smeared up to marked border 2023.

According to some embodiments, after using the optical model to identify the conductive paste edges, or couplant spreading edges, the electrical model is used, to identify the conductive paste edges, and a degree of compatibility between the optical model and the electrical model is estimated (using an algorithm executed by a processor such as processor 201). Ultimately, parameters of source localization are determined based on the degree of compatibility between the optical model and the electrical model. The term source localization refers to identifying where in the brain a particular type of activity originates based on the surface EEG recording. When the degree of compatibility between the optical model and the electrical model is high, it means the “true” electrode contact is established and the area/volume where electrode contact should be positioned may be defined with high degree of accuracy and a small confidence interval. When the degree of compatibility between the optical model and the electrical model is low it means that the electrode contact cannot be trusted (or cannot be highly trusted) for the purpose of source localization, and the area/volume where electrode contact should be positioned may not be defined with high degree of accuracy and therefore the confidence interval of the source localization increases. The area/volume of the electrode contact points positioning taking into account the confidence interval, may be defined as a “confidence volume” parameter. Other parameters of the source localization may also increase, for example, a parameter of confidence of direction of equivalent current dipole (ECD).

In other words, it can be said that the electrical model may be used as a quality check for the electrode contact of the electrode capsule. Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of non-transitory memory media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-31. (canceled)

32. A method for identifying a position of a plurality of electrodes in an electroencephalography (EEG) electrode array embedded to an EEG electrode array carrier when the EEG electrode array carrier is configured to be worn on a head of a subject, the method comprising:
capturing at least one image of three fiducials of the subject's head and at least two electrodes or visible marks around electrodes placed at known positions of the EEG electrode array carrier worn by the subject, using a stereo camera pair;
defining a coordinate system of the three fiducials and relating the at least two electrodes or visible marks around electrodes to the defined coordinate system;
determining the position of each of the plurality of electrodes on the head of the subject wearing the EEG electrode array carrier based at least on:
the defined coordinate system and triangulation of the three fiducials and at least two electrodes or visible marks around electrodes of the EEG electrode array carrier, and
a previously obtained 3D geometrical model of the subject's head and of the EEG electrode array, and a known mechanical model of the EEG electrodes' array carrier thereby, reconstructing a 3D geometrical model of the EEG electrode array embedded to the EEG electrode array carrier worn on the subject's head.

33. The method of claim 32, further comprising:
injecting current into one or more electrodes of the EEG electrode array, coupled to the head of the subject through a couplant;
measuring the voltage response on other electrodes of the EEG electrode array;
constructing a 3D electrical model of the EEG electrode array embedded to the EEG electrode array carrier worn on the subject's head;
constructing an electrical model of a couplant spreading based at least on:
the voltage response as a function of the shape of the couplant spreading of all the electrodes of the EEG electrode array over the subject's head surface, and
the 3D electrical model of the EEG electrode array embedded to the EEG electrode array carrier worn on the subject's head; and
identifying the weight center of the couplant coupling the electrodes, according to the electrical model of the couplant spreading, thereby identifying the position of the electrodes.

34. The method of claim 33, further comprising: coupling dry electrodes to the head of the subject and identifying the position of said dry electrodes according to a capacitive coupling function derived from the 3D electrical model of the EEG electrode array.

35. The method of claim 32, wherein capturing the at least one image of three fiducials and at least two electrodes or visible marks around electrodes placed at known positions of the EEG electrode array carrier is facilitated using a multi-view camera system, which is configured to capture at least one multi-view set of images at least at a single time moment, wherein said camera is a video camera or a still camera.

36. The method of claim 32, wherein the mechanical model of the EEG electrodes' array carrier, is un-stretchable, and preserves the geodetic distances between the electrodes, or is stretchable, with a known value of elasticity.

37. The method of claim 32, further comprising:
successively capturing one or more additional images of the head of the subject wearing the EEG electrode array carrier, so that at least three elements out of the three fiducials and the at least two electrodes or visible marks around electrodes in known positions of the EEG electrode array carrier, from the at least one image or from one of the multi-view set of images which are used as a reference image, are visible;
relating electrodes which are visible in the one or more additional images to the coordinate system defined in the reference image; and
triangulating other electrodes visible in the one or more additional images, thereby identifying the position of the electrodes visible in the one or more additional images.

38. The method of claim 33, wherein the electrical models of the EEG electrode array and of the couplant spreading are used as a reference for the reconstructed 3D geometrical model of the EEG electrode array, thereby constructing a 3D electro-geometrical model of the EEG electrode array embedded to the EEG electrode array carrier worn on the subject's head.

39. The method of claim 38, further comprising:
detecting a potential wrinkle in or a displacement of the EEG electrode array carrier on the subject's head by capturing one or more additional images of the head of the subject wearing the EEG electrode array carrier and/or by executing a machine learning algorithm by a processor, on the constructed 3D electro-geometrical model of the EEG electrode array; and
if a wrinkle or a displacement of the EEG electrode array carrier is detected, providing indication to the subject to realign the EEG electrode array carrier on their head or compensating for the wrinkle or displacement of the EEG electrode array carrier upon constructing the 3D electro-geometrical model of the EEG electrode array.

40. The method of claim 39, wherein the machine learning algorithm is a deep neural network trained on labeled database records of wrinkles.

41. The method of claim 37, wherein the successive capturing of the one or more additional images of the head of the subject wearing the EEG electrode array carrier is done when the subject's head is rotated relative to the position of the head when capturing the at least one image or when the stereo camera pair or multi-view camera system is rotated around the subject's head.

42. The method of claim 32, wherein the previously obtained 3D geometrical model of the subject's head is constructed according to at least one of the following: a set of photographs, video, Magnetic Resonance Imaging (MRI) scanning or Computed Tomography (CT) scanning of the subject's head and scanning of the subject's head with the EEG electrode array carrier.

43. A system for identifying a position of a plurality of electrodes in an EEG electrode array embedded to an EEG electrode array carrier when the EEG electrode array carrier is worn on a head of a subject, the system comprising:
a stereo camera pair, capturing at least one image of three fiducials of the subject's head and at least two electrodes or visible marks around electrodes placed at known positions of the EEG electrode array carrier worn by the subject;
a display for presenting EEG signals received from the EEG electrode array carrier and for locating the stereo camera pair; and
a processor, configured to execute a code for:
defining a coordinate system of the three fiducials and relating the at least two electrodes or visible marks around electrodes placed at known positions of the EEG electrode array carrier to the defined coordinate system; and
determining the position of each of the plurality of electrodes on the head of the subject wearing the EEG electrode array carrier based at least on:
the defined coordinate system and triangulation of the three fiducials and at least two electrodes or visible marks around electrodes of the EEG electrode array carrier, and
a previously obtained 3D geometrical model of the subject's head and of the EEG electrode array, and a known mechanical model of the EEG electrodes' array carrier thereby, reconstructing a 3D geometrical model of the EEG electrode array embedded to the EEG electrode array carrier worn on the subject's head.

44. The system of claim 43, further comprising:
an electrical power source for injecting current into one or more electrodes of the EEG electrode array, coupled to the head of the subject through a couplant;
a voltmeter for measuring the voltage response on other electrodes of the EEG electrode array;
a processor executing a code for:
constructing a 3D electrical model of the EEG electrode array embedded to the EEG electrode array carrier on the subject's head;
constructing an electrical model of a couplant spreading based at least on:
the voltage response as a function of the shape of the couplant spreading of all the electrodes of the EEG electrode array over the subject's head surface, or
the 3D electrical model of the EEG electrode array embedded to the EEG electrode array carrier worn on the subject's head; and
identifying the weight center of the couplant coupling the electrodes, according to the electrical model of the couplant spreading, thereby identifying the position of the electrodes.

45. The system of claim 44, further comprising capsules with a constant amount of couplant which are embedded to the EEG electrode array carrier wherein each capsule is located near one electrode of the EEG electrode array.

46. The system of claim 45, wherein electrodes of the EEG electrode array are located inside the capsules, as electrode-capsule, and when a capsule is opened, the couplant inside the capsule is configured to be placed in a space between the electrode inside the capsule and the head of the subject.

47. The system of claim 44, further comprising electrode-stickers which are embedded to the EEG electrode array carrier wherein the electrode-capsules are configured to be coupled to locations with hair on the subject's head and the electrode-stickers are configured to be coupled to locations without hair.

48. The system of any of claim 47, wherein the EEG electrode array carrier used with the capsules or electrode-capsules is a transparent cap or a cap with one or more transparent windows.

49. The system of claim 43, wherein the stereo camera pair is configured to capture at least one image of the subject's head wearing the transparent cap or the cap with one or more transparent windows after the couplant is smeared on the subject's head, and the processor executes a code for determining couplant edges based on the at least one image captured.

50. The system of claim 49, wherein couplant edges are determined based on electrically measured intercontact distances, and a degree of compatibility between the couplant edges determined based on the at least one image and the couplant edges determined based on the electrically measured intercontact distances, is estimated.

51. The system of claim 50, wherein parameters of source localization are determined based on the degree of compatibility between the couplant edges determined based on the at least one image and the couplant edges determined based on the electrically measured intercontact distances.
